(19) United States
(12) Patent Application Publication
(10) Pub. No.: US 2004/0018250 A1
(43) Pub. Date: Jan. 29, 2004

(54) Methods for preparing high pressure/high shear dispersions containing waxes and other semi-solids and oils

(76) Inventors: Joseph D. Ceccoli, Farmingville, NY (US); Michael Ross, Dix Hills, NY (US); James M. Wilmott, Shoreham, NY (US); Todd Coleman, Boswell, PA (US); Timothy K. Crawford, Oxnard, CA (US)

Correspondence Address: Robert C Sullivan, Darby & Darby, Post Office Box 5257, New York, NY, 10150-5257, US

(21) Appl. No.: 10/332,395

(22) PCT Filed: Jul. 11, 2001
(86) PCT No.: PCT/US01/21746

(51) Int. Cl. A61K 35/78

(52) U.S. Cl. 424/725

(57) ABSTRACT

The present invention provides an oil-in-water wax dispersion comprising one or more waxes or hydrophobic semi-solids, a dispersion initiator, optionally oen or more plasticizing agents or solvents and/or co-solvents, and water. The invention also provides a method for preparing these oil-in-water wax dispersions and their use in topical, oral anal, ophthalmic, vaginal, otic and nasal formulations.

### FIELD OF THE INVENTION

The present invention relates to an oil-in-water dispersion composed of one or more waxes or hydrophobic semi-solids, a dispersion initiator, optionally one or more plasticizing agents or solvents and/or co-solvents, and water. The invention also describes a method for preparing these oil-in-water dispersions and their use in topical, oral, anal, ophthalmic, vaginal, otic and nasal formulations.

Elements of the invention are described in the ensuing text. This description is provided as information to help define the extent of the invention and is non-limiting in nature.

### BACKGROUND OF THE INVENTION

Waxes are commonly used in formulations for topically applied pharmaceutical or cosmetic products for their relatively high water resistance, their ability to modify the textural, Theological and tactile properties of products. Waxes provide a richer feel to products during the application process and provide afterfeel properties ranging from smooth and soft to a heavy coated feel.

In an effort to effectively incorporate waxes or other hydrophobic semi-solids into topical preparations, waxes are typically either solubilized in a suitable hydrophobic fluid, and/or are incorporated into water-in-oil (W/O) or oil-in-water (O/W) emulsions. However, emulsions present problems both in the conditions used for processing and in the selection of ingredients used to form the emulsions.

Emulsions are prepared by adding emulsifiers or surfactants to the hydrophobic and hydrophilic phases of a composition to reduce the surface tension of water and the interfacial tension between the hydrophobic and hydrophilic phases. The hydrophilic phase, containing water and water compatible emulsifiers and components, is prepared and heated with mixing to a temperature in excess of 70° C. A hydrophobic phase containing the waxes and other non-polar ingredients such as oils, and oil compatible emulsifiers and components, is heated to temperatures sufficient to melt the wax and mixed until a homogeneous preparation results. The hydrophobic phase is added to the hydrophilic phase and mixed with suitable agitation to intimately intermingle the phases. The total composition is then cooled to remove the excess heat until the composition reaches ambient temperature.

Standard emulsion preparations are costly to manufacture due to a variety of factors including the high amount of energy required to heat the preparation, the specialized equipment required to process the emulsion such as specialized pumps and cooling/heating equipment and longer processing times.

Emulsifiers or surfactants also unfortunately reduce the water resistivity of the formulations because surfactants (and other emulsifiers) facilitate the removal of the formulations from the surface of the skin when exposed to water.

Surfactants also strip protective layers from the lipid barrier of the skin or the lipid bilayer of epithelial cell membranes leaving the skin tissue vulnerable to further injury. Thus, the surfactants themselves can cause irritation or the damaged barrier will permit the passage of other materials that can cause irritation or increase skin sensitivity and allergic reactions (see, e.g., Effendy I, Maibach H I, Contact Dermatitis 1995 October; 33(4);217-25; Barany E, Lindberg M, Loden M, Contact Dermatitis 1999 February;40(2):98-103).

Accordingly, it is desirable to produce a stable topical formulation comprising a surfactant-free oil-in-water dispersion of a wax or hydrophobic semi-solid that is water resistant and provides aesthetically pleasing tactile properties.

### SUMMARY OF THE INVENTION

The present invention provides a method for preparing a surfactant-free oil-in-water wax dispersion comprising a hydrophobic phase comprising a wax, mixed with an aqueous phase. In one embodiment the hydrophobic phase further comprises one or more hydrophobic components which may include a plasticizing agent, a solvent, and a co-solvent. The hydrophobic phase may further comprise other active agents. The aqueous phase may comprise in addition to water, water compatible additives and or modifiers. In one embodiment, the aqueous phase further comprises a dispersion initiator.

The method of the invention comprises the steps of first mixing the hydrophobic phase comprising a wax, and optionally other hydrophobic components, and then mixing the hydrophobic phase with the aqueous phase, and subjecting the hydrophobic/aqueous mixture to high pressure, high shear or high pressure/high shear mixing conditions to form a stable oil-in-water wax dispersion.

The hydrophobic phase is present in an amount of from 1 to 60 wt % of the total dispersion and preferably from 5 to 40 wt %. The aqueous phase is present in an amount of from 40 to 99 wt % of the total dispersion and preferably from 60 to 95 wt %.

Preferably, the wax is shea butter, cocoa butter, carnula wax, petrolatum, castor wax, chamomile wax, bayberry wax, candelilla wax, carnauba wax, clary wax, beeswax, orange peel wax, Japan wax, mink wax, rice bean wax, oricury wax, palm kernel wax, raspberry leaf wax, rosemary flower wax, sweet violet leaf wax, tuberose flower wax, ylang ylang flower wax, everlasting wax, myrtle leaf wax, spermaceti wax, jojoba wax, jotoba butter waxes, and combinations thereof; ceresine wax, ozokerite, parafin wax and microcrystaline wax; synthetic beeswax, synthetic candelilla wax, synthetic carnauba wax, or synthetic spermaceti; cetyl demithicone, dialkyldimethyl polysiloxane, dimethiconol hydroxystearate, dimethiconol stearate, stearoxymethicone/dimethicone copolymer and stearoxytrimethylsilane. The wax is present in an amount of from 1 to 50 wt % of the total dispersion and preferably from 5 to 40 wt %.

Additional hydrophobic components are selected from the group consisting of hydrogenated castor oil, hydrogenated cottonseed oil, hydrogenated jojoba oil, hydrogenated palm kernel oil, hydrogenated rapeseed oil and hydrogenated rice bran wax.

The dispersion initiator is preferably phospholipid, lecithin, or combinations thereof. The dispersion initiator is present in an amount of from 0.01 to 10.0 wt % of the total dispersion and preferably from 0.1 to 5.0 wt %.

The plasticizing agent is preferably cyclomethicone, hydrogenated polyisobutene, polyethylene glycol fluid or butylene glycol. The plasticizing agent is present in an amount of from 0.5 to 30 wt % of the total dispersion and preferably from 1.0 to 20 wt %.

The solvent and co-solvent are preferably silicone or silicon derivatives such as cyclomethicone; hydrogenated polyisobutene; polymers of ethylene oxides such as polyethylene glycol; or butylene glycol. The solvent is present in an amount of from 5.0 to 50 wt % of the total dispersion and preferably from 10 to 40 wt %. The co-solvent is present in an amount of from 1.0 to 25 wt % of the total dispersion and preferably from 2 to 20 wt %.

In another aspect the present invention provides an oil in water wax dispersion prepared by the method of the invention. The dispersion comprises a hydrophobic phase comprising a wax, mixed with an aqueous phase. In one embodiment the hydrophobic phase further comprises one or more hydrophobic components which may include a plasticizing agent, a solvent, and a co-solvent. The hydrophobic phase may further comprise other active agents. The aqueous phase may comprise in addition to water, water compatible additives and or modifiers. In one embodiment, the aqueous phase further comprises a dispersion initiator.

The hydrophobic phase is present in an amount of from 1 to 60 wt % of the total dispersion and preferably from 5 to 40 wt %. The aqueous phase is present in an amount of from 40 to 99 wt % of the total dispersion and preferably from 60 to 95 wt %.

Preferably, the wax is shea butter, cocoa butter, carnula wax, petrolatum, castor wax, chamomile wax, bayberry wax, candelilla wax, carnauba wax, clary wax, beeswax, orange peel wax, Japan wax, mink wax, rice bean wax, oricury wax, palm kernel wax, raspberry leaf wax, rosemary flower wax, sweet violet leaf wax, tuberose flower wax, ylang ylang flower wax, everlasting wax, myrtle leaf wax, spermaceti wax, jojoba wax, jotoba butter waxes, and combinations thereof; ceresine wax, ozokerite, parafin wax and microcrystaline wax; synthetic beeswax, synthetic candelilla wax, synthetic carnauba wax, or synthetic spermaceti; cetyl demithicone, dialkyldimethyl polysiloxane, dimethiconol hydroxystearate, dimethiconol stearate, stearoxymethicone/dimethicone copolymer and stearoxytrimethylsilane. The wax is present in an amount of from 1 to 50 wt % of the total dispersion and preferably from 5 to 40 wt %.

Additional hydrophobic components are selected from the group consisting of hydrogenated castor oil, hydrogenated cottonseed oil, hydrogenated jojoba oil, hydrogenated palm kernel oil, hydrogenated rapeseed oil and hydrogenated rice bran wax.

The dispersion initiator is preferably phospholipid, lecithin, or combinations thereof. The dispersion initiator is present in an amount of from 0.01 to 10.0 wt % of the total dispersion and preferably from 0.1 to 5.0 wt %.

The plasticizing agent is preferably cyclomethicone, hydrogenated polyisobutene, polyethylene glycol fluid or butylene glycol. The plasticizing agent is present in an amount of from 0.5 to 30 wt % of the total dispersion and preferably from 1.0 to 20 wt %.

The solvent and co-solvent are preferably silicone or silicon derivatives such as cyclomethicone; hydrogenated polyisobutene; polymers of ethylene oxides such as polyethylene glycol; or butylene glycol. The solvent is present in an amount of from 5.0 to 50 wt % of the total dispersion and preferably from 10 to 40 wt %. The co-solvent is present in an amount of from 1.0 to 25 wt % of the total dispersion and preferably from 2 to 20 wt %.

In a still further aspect, the present invention provides a composition comprising a) a dispersion prepared by the method of the invention; and b) a base composition comprising water and a hydrophilic rheological modifying agent. According to this embodiment of the invention, the composition comprises the dispersion of a mixture of a hydrophobic phase comprising a wax and an aqueous phase which has been subjected to high pressure, high shear, or high pressure/high shear conditions until a dispersion is obtained. The dispersion is then mixed with the base composition to form the composition of the invention. In one embodiment the hydrophobic phase of the dispersion further comprises one or more hydrophobic components. Hydrophobic components may include a plasticizing agent, a solvent and a co-solvent. The hydrophobic phase may further comprise other active agents.

The aqueous phase of the dispersion may further comprise thickeners, a dispersion initiator and one or more active agents.

The dispersion is present in an amount of from about 0.1 to about 80 wt % and preferably from about 0.5 to about 40 wt % of the composition. The base composition is present in an amount of from about 20 to 99 wt % of the composition.

The base composition typically comprises from about 0.001 to about 50% and preferably from about 0.01 to about 10%, and more preferably from about 0.1 to about 5% by weight of hydrophilic Theological modifying agents. The base composition typically comprises from about 0.001 to about 99.99%, preferably from about 1 to about 99.99%, and more preferably from about 20 to about 99.99% by weight of water.

The composition may further include other surfactant-free oil-in-water dispersions or other additives/modifiers such as are required for the final product.

The composition may be prepared by mixing the dispersion with the aqueous base composition using methods known in the art.

Suitable hydrophilic rheological modifying agents for the base composition include hydrophilic gelling agents including carboxyvinyl polymers, acrylic copolymers, polyacrylamides, polysaccharides, natural gums and clays, or phosphorylated starch derivatives.

The composition may further comprise other additives such as physiological actives and/or aesthetic modifiers such as fragrance, chelating agents, colorants and antioxidants as required or suitable for the preparation of topical compositions for the treatment of dermal, otic, anal, oral, vaginal, nasal and opthalmic disorders. Such additives are typically mixed with the dispersion and other ingredients using processes and equipment known in the art.

In one embodiment, the wax is first heated and melted with the plasticizing agent and one or more solvents and co-solvents, mixed with the aqueous composition comprising a dispersion initiator, and then passed through high pressure and high shear processing to produce an oil in water dispersion of a controlled particle size and uniformity. The resulting high pressure and high shear dispersion compositions can be used for prepare a topical composition by the dispersion with low shear in the surfactant-free base composition. The resulting composition may be in the form of a lotion, cream, paste, serum or spray.

### DETAILED DESCRIPTION OF THE INVENTION

The method of the present invention forms a stable surfactant-free dispersion comprising one or more waxes or other hydrophobic semi-solids, optionally plasticizing agents and/or solvents or co-solvents, and an aqueous phase optionally comprising a dispersion inhibitor. The dispersions formed by the method of the invention have particle size in the range of from about 0.1 to 5.0 microns. The dispersions may be used in topical formulations for cosmetic or pharmaceutical applications, in the form of a lotion, cream, paste, serum or spray. These formulations have a more emollient and luxurious feel, and have a reduced irritation potential compared to prior art formulations.

Definitions

All patents, applications, test methods and publications referenced in this specification are hereby incorporated by reference in their entirety. In case of conflict, the present description will prevail.

As used herein, the term “wax”, unless specified otherwise, means a hydrophobic semi-solid material, as defined herein below.

As used herein the term “surface-active” or “surface-active agent” refers to a substance capable of reducing the surface tension of a liquid in which it is dissolved.

A “non-surface active agent” is a substance which does not significantly reduce the surface tension of a liquid in which it is dissolved or dispersed.

As used herein, the term “surfactant” refers to a surface-active substance.

As used herein, the term “surfactant-free” refers to dispersions or compositions that are produced without the use of surface-active ingredients. The composition preferably comprises less than about 3% by weight and more preferably less than about 1% by weight of emulsifying surfactants, based upon 100% weight of total composition.

The terms “dispersion” or “oil-in-water dispersion” are used interchangeably herein, and refer to the suspension of an oil (or nonpolar substance) or hydrophobic phase or component in a polar (for example, aqueous) composition.

The term “dispersion initiator” refers to a material that facilitates the mixing of the hydrophobic and hydrophilic phases, i.e., agents that help to initiate the micellularization process. These agents may be present in an amount of from about 0.01 to about 5.0 wt % of the total dispersion. Suitable agents include phospholipids which have low dissociation constants of about in the range of about of 10⁻¹⁰ to 10⁻³⁰ M, and at low oil concentrations do not form micelles, but form very stable lipid bilayers. Phospholipids also appear to increase the surface tension of the composition when drying. A composition prepared using a dispersion initiator is considered to be substantially free of emulsifying surfactants.

A plasticizing agent renders a material to which it is added softer, more pliable or more malleable.

Hydrophobic Semi-Solids

Hydrophobic semi-solids are hydrophobic compounds which produce a solid, semi-solid or paste consistency at 25° C. and which melt at a temperature of between about 27° C. and 150° C. Hydrophobic semi-solids suitable for the present invention include hydrocarbons, modified hydrocarbons, silicones or hydrocarbon derivatives of silicone. Preferred hydrophobic semi-solids include waxes, cocoa butter and shea butter.

Waxes of the present invention are composed of one or more saturated, unsaturated, linear, branded or cyclic hydrocarbon chains. An example of a suitable hydrocarbon compounds are illustrated by the following formula:

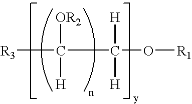

wherein n=0 to 100; y=0 or 1; and R₁, R₂, R₃=H or a saturated, unsaturated, linear, branched or cyclic alkyl or alkyloyl radical of from about 1 to about 100 carbon atoms.

Eutectic blends of these compounds can also be combined to produce other waxes, greases or other hydrophobic semi-solids. Examples of such blends include, but are not limited to cocoa butter, shea butter, castor wax, chamorrile wax, bayberry wax, candelilla wax, carnauba wax, clary wax, beeswax, orange peel wax, Japan wax, mink wax, rice bean wax, oricury wax, palm kernel wax, raspberry leaf wax, rosemary flower wax, sweet violet leaf wax, tuberose flower wax, ylang ylang flower wax, everlasting wax, myrtle leaf wax, spermaceti wax, jojoba wax, joBoba butter waxes. Waxes can be produced by reducing or partially reducing unsaturated fatty acids or esters. Examples of such waxes include but are not limited to: hydrogenated castor oil, hydrogenated cottonseed oil, hydrogenated jojoba oil, hydrogenated palm kernel oil, hydrogenated rapeseed oil and hydrogenated rice bran wax.

Other waxes are derived from petroleum and include, but are not limited to ceresine wax, ozokerite, paraffin wax and microcrystalline wax. Waxes can be formed naturally such as beeswax or their components can be prepared synthetically such as synthetic beeswax, synthetic candelilla wax, synthetic carnauba wax, or synthetic spermaceti. Waxes can also be derivatized with silicone fractions to modify their properties. Examples include cetyl demithicone, dialkyldimethyl polysiloxane, dimethiconol hydroxystearate, dimethiconol stearate, stearoxymethicone/dimethicone copolymer and stearoxytrimethylsilane.

Dispersion Initiators

Suitable dispersion initiators include phospholipids and lecithins.

Phospholipids, which have a critical micelle concentration (cmc) of the order of 10⁻¹⁰ to 10⁻³⁰ M, do not exist as free molecules in water based dispersions, but form very stable lipid bilayers. Surfactants, on the other hand, which have a cmc in the range of 0.1 to 30 M, when dispersed in water merely reduce the surface tension of the water. The high stability of lipids in self-assembled microstructures is very important in the formation of surfactant-free emulsions.

In particular embodiments, the dispersion may comprise from 0.01% to 8% by weight (preferably 0.01 to 5% by weight) of one or more non-surfactant phospholipids. Exemplary phospholipids include Phospholipon 80, 80H (American Lecithin Co., Oxford, Conn.), Basis LP-2OH (Ikeda Corp., Japan), and Catemol, a fatty acid of quaternary amines (Phoenix Chemicals Inc., Somerville, N.J.).

The dispersion may also include one or more lecithins, for example Alcolec® (American Lecithin, Oxford, Conn.).

Solvents and Co-solvents

For the purposes of the present invention, a solvent is defined as a fluid or solid into which one or more waxes or hydrophobic semi-solids are dissolved. A co-solvent is a second solvent added to the original solvent to increase the dissolution of the wax or hydrophobic semi-solid. Amounts and selections of solvent and, if desired co-solvents, appropriate for the dispersions of the invention are readily determined in order to facilitate the dissolution of the wax. The solvents and co-solvents used in the present invention can be, but are not limited to, low polar to non-polar materials. Preferably, the solvent is a fluid at ambient temperature but can be a solid such as a wax, grease or paste which has been converted to a fluid by heating to a temperature exceeding its melting point before the addition of the solvent.

Suitable solvents and co-solvents that may be used in the present invention have the following formula:

CₙH(2n+2−x)

wherein n is an integer from 6 to greater than 1 million and x is 0 or an even integer no greater than n.

These materials may include saturated, unsaturated, branched and cyclic hydrocarbon chains. Examples of solvents and co-solvents include mineral oil, petrolatum, polybutylenes and hydrogenated polyisobutene.

Other useful solvents and co-solvents are the mono, di, tri or poly-branches, -cyclic, -saturated or unsaturated alkyl esters or ethers of di, tri or polyhydroxy compounds such as ethylene glycol, propylene glycol, glycerin, sorbitol or polyol. An example of a preferred material is a propylene glycol monoisostearate.

Other useful solvents and co-solvents include, but are not limited to the following: branched, saturated or unsaturated vegetable oils such as soybean oil, babassu oil, castor oil, cottonseed oil, Chinese tallow oil, crambe oil, perilla oil, Danish rapeseed oil, rice bran oil, palm oil, palm kernel oil, olive oil, linseed oil, coconut oil, sunflower oil, safflower oil, peanut oil, and corn oil. Preferred saturated and unsaturated vegetable oils are those having fatty acid components with 6 to 24 carbon atoms.

Additional solvents and co-solvents include esters of the type:

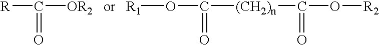

wherein R₁ and R₂ are saturated, unsaturated, branched or cyclic alkyl radicals of 2 to 24 carbon atoms and n is an integer from 0 to about 20. Suitable esters include isopropyl palmitate and diisopropyl adipate.

Other suitable solvents and co-solvents that can be used in the current composition are the type:

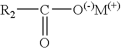

wherein R₁ is a saturated, unsaturated, branched or cyclic alkyl radical from C₂ to C₂₄; M⁽⁺⁾ is defined by NR₂R₃R₄R₅, wherein R₂, R₃ and R₄ are hydrogen or a saturated, unsaturated or branched alkyl or hydroxalkyl radical from C₁ to C₁₀; R₅ is a saturated, unsaturated, branched or cyclic alkyl or substituted alkyl radical from C₂ to C₂₄. An example of the above is lauramine oleate.

Another class of suitable solvents and co-solvents is formed by the polymerization of alkylene oxide monomers of the formula:

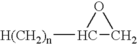

wherein n is an integer from 0 to about 3.

These materials can be either a homogenous polymer or a copolymer of two or more monomers. Examples of homogeneous polymers include polyethylene oxide, polypropylene oxide and polybutylene oxide. Typically, the molecular weights of these materials are between 100 and 10,000 daltons. Preferred solvents or co-solvents include PEG-4 and PEG-8 (Dow Chemical). Additionally, these materials can be condensed with a mono or polyhydroxyalkyl alcohol as exemplified by the UCON fluids from the Dow Chemical Company.

The solvent and co-solvents are preferably silicone and/or silicone derivatives. Preferably, the silicone is insoluble in water. Suitable water-insoluble silicone materials include, but are not limited to, cyclomethicone, polyallylsiloxanes, polyarylsiloxanes, polyalkylarylsiloxanes, polysiloxane gums and polyethersiloxane copolymers. Examples of suitable silicone materials are disclosed in U.S. Pat. Nos. 4,788,006; 4,341,799; 4,152,416; 3,964,500; 3,208,911; 4,364,837 and 4,465,619, all of which are incorporated herein by reference.

Exemplary silicone and silicone derivatives include branched or linear cyclical silicone or silicone derivatives, cyclomethicone, dimethicone polysiloxane, dimethiconol, polysiloxanes, polysiloxane copolymers, polyalkyl aryl silanes, polyaryl siloxanes, and polyalkyl siloxanes. Preferred examples of silicone solvents or co-solvents include: low viscosity dimethicone, phenyl trimethicone (Dow Corning) and silicone fluid DC 345 (Dow Corning).

Plasticizers

Low polar or non-polar fluids described in the section of solvents and co-solvents are suitable as plasticizing agents. Preferred plasticizing agents include cyclomethicone, hydrogenated polyisobutene, polyethylene glycol fluid and butylene glycol.

Preservatives

Suitable preservatives include, but are not limited to, chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol, methylchloroisothiazolinone, methylisothiazolinone, and any combination of any of the foregoing. In certain embodiments, compositions of the invention may comprise preservatives such as Phenonip (NIPA Laboratories, Wilmington, Del.); Germazide™ MPB (Collaborative Laboratories, Inc. East Setauket, N.Y.); and polyoxethylene ethers.

Base Composition

Rheological modifying agents, also referred to herein as thickeners, within the scope of the invention include any substance which increases or decreases the viscosity of the wax containing composition. Suitable Theological modifying agents include, but are not limited to, phosphorylated starch derivatives, carbohydrate based Theological modifying agents, polymeric and copolymeric Theological modifying agents, inorganic Theological modifying agents, protein rheological modifying agents, polypeptide Theological modifying agents, and any combination of any of the foregoing.

The term “phosphorylated starch derivative” includes, but is not limited to, starches containing a phosphate group. Suitable phosphorylated starch derivatives include, but are not limited to, hydroxyalkyl starch phosphates, hydroxyalkyl distarch phosphates, and any combination of any of the foregoing. Non-limiting examples of hydroxyalkyl starch phosphates and hydroxyalkyl distarch phosphates include hydroxyethyl starch phosphate, hydroxypropyl starch phosphate, hydroxypropyl distarch phosphate (including sodium hydroxypropyl starch phosphate), and any combination of any of the foregoing.

Non-limiting examples of suitable carbohydrate based rheological modifying agents include algin and derivatives and salts thereof, such as algin, calcium alginate, propylene glycol alginate, and ammonium alginate; carrageenan (Chondrus crispus) and derivatives and salts thereof, such as calcium carrageenan and sodium carrageenan; agar; cellulose and derivatives thereof, such as carboxymethyl hydroxyethylcellulose, cellulose gum, cetyl hydroxyethylcellulose, hydroxyethylcellulose, hydroxypropylcellulose, hydroxypropyl methylcellulose, methylcellulose, ethylcellulose, and cellulose gum; chitosan and derivatives and salts thereof, such as hydroxypropyl chitosan, carboxymethyl chitosan, and chitin; gellan gum; guar (Cyanopsis tetragonoloba) and derivatives thereof, such as guar hydroxypropyltrimonium chloride and hydroxypropyl guar; hyaluronic acid and derivatives thereof, such as sodium hyaluronate; dextran and derivatives thereof; dextrin; locust bean (Ceratonia siliqua) gum; starches, such as starch polyacrylonitrile copolymer-potassium salt and starch polyacrylonitrile copolymer-sodium salt; pectin; sclerotium gum; tragacanth (Astragalus gummifer) gum; xantham gum and derivatives thereof; and any combination of any of the foregoing.

Non-limiting examples of suitable polymeric and copolymeric rheological modifing agents include acrylates, methacrylates, polyethylene and derivatives thereof, and any combination of any of the foregoing. Suitable acrylates and methacrylates include, but are not limited to, carbomer and derivatives and salts thereof, acrylate/C₁₀-C₃₀ alkyl acrylate crosspolymer, acrylate/ceteth-20 itaconate copolymer, acrylate/ceteth-20 methacrylate copolymers, acrylate/steareth-20 methacrylate copolymers, acrylate/steareth-20 itaconate copolymers, acrylate/steareth-50 acrylate copolymers, acrylate/VA crosspolymers, acrylate/vinyl isodecanoate crosspolymers, acrylic acid/acrylonitrogen copolymers, ammonium acrylate/acrylonitrogen copolymers, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylate/acrylamide copolymer, acrylate copolymers, acrylate/hydroxyester acrylate copolymers, acrylate/octylarylamide copolymers, acrylate/PVP copolymers, AMP/acrylate copolymers, butylester of PVM-MA copolymer, carboxylate vinylacetate terpolymers, diglycol/CHDM/isophthalates/SIP copolymer, ethyl ester of PVM-MA copolymer, isopropyl ester of PVM-MA copolymer, octylacrylamide/acrylate/butylaminoethyl methacrylate copolymers, polymethacrylamidopropyltrimonium chloride, propylene glycol oligosuccinate, polyvinylcaprolactam, PVP, PVP/dimethylaminoethylmethacrylate copolymer, PVP/DMAPA acrylate copolymers, PVP/carbamyl polyglycol ester, PVP/VA copolymer, PVP/VA vinyl propionate copolymer, PVP/vinylcaprolactam/DMAPA acrylate copolymers, sodium polyacrylate, VA/butyl maleate/isobornyl acrylate copolymers, VZ/crotonates copolymer, VA/crotonates vinyl neodecanoate copolymer, VA crotonates/vinyl propionate copolymer, vinyl caprolactam/PVP/dimethylaminoethylmethacrylate copolymer, and any combination of any of the foregoing.

Non-limiting examples of suitable inorganic thickening agents include clays and derivatives thereof, silicates, silicas and derivatives thereof, and any combination of any of the foregoing. Suitable clays and derivatives thereof include, but are not limited to, bentonite and derivatives thereof, such as quaternium-18 bentonite; hectorite and derivatives thereof, such as quaternium-18 dectorite; montmorillonite; and any combination of any of the foregoing. Suitable silicates include, but are not limited to, magnesium aluminum silicate, sodium magnesium silicate, lithium magnesium silicate, tromethamine magnesium aluminum silicate, and any combination of any of the foregoing. Suitable silicas and derivatives thereof include, but are not limited to, hydrated silica, hydrophobic silica, and any combination of any of the foregoing.

Suitable protein and polypeptide rheological modifying agents include, but are not limited to, proteins and derivatives and salts thereof, polypeptides and derivatives and salts thereof, and any combination of any of the foregoing. Non-limiting examples of protein and polypeptide Theological modifying agents include albumin, gelatin, keratin and derivatives thereof, fish protein and derivatives thereof, milk protein and derivatives thereof, wheat protein and derivatives thereof, soy protein and derivatives thereof, elastin and derivatives thereof, silk protein and derivatives thereof, and any combination of any of the foregoing.

Preferred Theological modifying agents include, but are not limited to, carbomer, acrylate/alkyl acrylate crosspolymers, acrylate/vinyl isododecanoate crosspolymer, xantham gum, locust bean gum, guar gum, and any combination of any of the foregoing. A more preferred combination of Theological modifying agents comprises carbomer and an acrylate/alkyl acrylate copolymer, such as an acrylate/C₁₀-C₃₀ alkyl acrylate crosspolymer. According to the International Cosmetic Ingredient Dictionary and Handbook (7^{th} Ed., The Cosmetic, Toiletry, and Fragrance Association), carbomer is a homopolymer of acrylic acid crosslinked with an allyl ether of pentaerythritol, an allyl ether of sucrose, or an allyl ether of propylene. The term “acrylate/alkyl acrylate crosspolymer” includes, but is not limited to, copolymers of alkyl acrylates with one or more monomers of acrylic acid, methacrylic acid, or one of their short chain (i.e. C₁₋₄ alcohol) esters, wherein the crosslinking agent is, for example, an allyl ether of sucrose or pentaerytritol. Preferably, the alkyl acrylates are C₁₀-C₃₀ alkyl acrylates. Examples of such copolymers include, but are not limited to, those commercially available as Carbopol™ 1342, Carbopol™ 1382, Pemulen™ TR-1, and Pemulen™ TR-2, from Goodrich Specialty Chemicals of Cleveland, Ohio.

Preferred Theological modifying agents include, but are not limited to hydrophilic gelling agents, such as carboxyvinyl polymers (carbomer), acrylic copolymers (e.g. acrylate/alkyl acrylate copolymers), polyacrylamides, polysaccharides (e.g. hydroxypropylcellulose), natural gums, clays, and any combination of any of the foregoing.

The base composition typically comprises from about 0.001 to about 50% and preferably from about 0.01 to about 10%, and more preferably from about 0.1 to about 5% by weight of Theological modifying agents. The base composition typically comprises from about 0.001 to about 99.99%, preferably from about 1 to about 99.99%, and more preferably from about 20 to about 99.99% by weight of water.

Active Agents

Suitable active agents include, but are not limited to, anti-acne agents, antimicrobial agents, anti-inflammatory agents, analgesics, antierythemal agents, antipruritic agents, antiedemal agents, antipsoriatic agents, antifungal agents, skin protectants, sunscreen agents, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, amino acids, peptides, minerals, ceramides, biohyaluronic acids, enzymes and any combination of any of the foregoing.

Preferred anti-acne agents include, but are not limited to, salicylic acid, retinoic acid, alpha hydroxy acid, benzoyl peroxide, sodium sulfacetamide, clindamycin, and any combination of any of the foregoing. Preferred combinations of anti-acne agents to be incorporated in the composition include salicylic acid, retinoic acid, and hydrocortisone; sodium sulfacetamide and clindamycin; salicylic acid and clindamycin; salicylic acid, alpha hydroxy acid, and tetrahydrozoline.

Suitable antimicrobial agents include, but are not limited to, benzalkonium chloride, benzethonium chloride, chlorhexidine gluconate, chloroxylenol, cloflucarban, fluorosalan, hexachlorophene, hexylresorcinol, iodine complex, iodine tincture, para-chloromercuriphenol, phenylmercuric nitrate, thimerosal, vitromersol, zyloxin, triclocarban, triclosan, methyl-benzethonium chloride, nonyl phenoxypoly(ethyleneoxy) ethanol-iodine, para-chloro-meta-xylenol, providone-iodine complex, poloxamer-iodine complex, triclorcarban, undecoylium chloride-iodine complex, and any combination of any of the foregoing.

Suitable anti-inflammatory agents include, but are not limited to, alidoxa, allantoin, aloe vera, aluminum acetate, aluminum hydroxide, bismuth subnitrate, boric acid, calamine, casein, cellulose, microporous, cholecatciferol, cocoa butter, cod liver oil, colloidal oatmeal, cysteine hydrochloride, dexpanthenol, dimethicone, glycerin, kaolin, lanolin, live yeast cell derivative, mineral oil, peruvian balsam, petrolatum, protein hydrolysate, racemethionine, shark liver oil, sodium bicarbonate, sulfur, talc, tannic acid, topical starch, vitamin A, vitamin E, white petrolatum, zinc acetate, zinc carbonate, zinc oxide, hydrocortisone, betamethasone, ibuprofen, indomethicin, acetyl salicylic acid, tacrolimus, flucoinolone acetonide, sodium sulfacetamide, and any combination of any of the foregoing.

Suitable analgesics include, but are not limited to, diphenhydramine, tripeiennamine, benzocaine, dibucaine, lidocaine, tetracaine, camphor, menthol, phenol, resorcinol, matacresol, juniper tar, methyl salicylate, turpentine oil, capsicum, methyl nicotinate, , β-glucan, and any combination of any of the foregoing.

Suitable antierythemal agents include, but are not limited to, tetrahydrozoline and hydrocortisone.

Suitable antipruritic agents include, but are not limited to, diphenhydramine, pramoxine, antihistamines, and any combination of any of the foregoing.

Suitable antiedemal agents, include, but are not limited to, pregnenalone acetate, tannin glycosides, and any combination of any of the foregoing.

Suitable antipsoriatic agents include, but are not limited to, caleipotriene, coal tar, anthralin, vitamin A, and any combination of any of the foregoing. Preferred combinations of antipsoriatic agents include, but are not limited to, hydrocortisone, retinoic acid, and alpha hydroxy acid; dovonex, salicylic acid, and a sunscreen agent; indomethacin, salicylic acid, and urea; anthralin and salicylic acid; and anthralin and indomethacin. Other suitable antipsoriatic agents include, but are not limited to, caleipotriene, coal tar, anthralin, vitamin A, and any combination of any of the foregoing.

Suitable antifungal agents include, but are not limited to, clioquinol, haloprogin, miconazole nitrate, clotrimazole, metronidazole, tolnaftate, undecylenic acid, iodoquinol, and any combination of any of the foregoing.

Suitable skin protectants include, but are not limited to, cocoa butter, dimethicone, petrolatum, white petrolatum, glycerin, polyphosphorylcholine glycol acrylate, shark liver oil, allantoin, and any combination of any of the foregoing.

Suitable sunscreen agents include, but are not limited to, ethylhexyl methoxycinnamate, avobenzone, benzophenone-3, octocrylene, titanium dioxide, zinc oxide, and any combination of any of the foregoing.

Suitable antioxidants include, but are not limited to, scavengers for lipid free radicals and peroxyl radicals, quenching agents, and any combination of any of the foregoing. Suitable antioxidants include, but are not limited to, tocopherol, BHT, beta-carotene, vitamin A, ascorbic acid, ubiquinol, ferulic acid, azelaic acid, thymol, catechin, sinapic acid, EDTA, lactoferrin, rosmariquinone, hydroxytyrosole, sesamol, 2-thioxanthine, nausin, malvin, carvacone, chalcones, glutathione isopropyl ester, xanthine, melanin, guanisone, lophorphyrins, 8-hydroxyxanthine, 2-thioxanthione, vitamin B₁₂, plant alkaloids, catalase, quercetin, tyrosine, SOD, cysteine, methionine, methylsulphonylmethane (MSM), genistein, NDGA, procyanidin, hamamelitannin, ubiquinone, trolox, licorice extract, propyl gallate, sinapic acid, and any combination of any of the foregoing.

Suitable vitamins include, but are not limited to, vitamin E, vitamin A palmitate, vitamin D, vitamin F, vitamin B₆, vitamin B₃, vitamin B₁₂, vitamin C, ascorbyl palmitate, vitamin E acetate, biotin, niacin, DL-panthenol, magnesium ascorbyl phosphate and any combination of any of the foregoing.

Suitable amino acids include, but are not limited to, glycine, serine, and any combination of any of the foregoing.

Aesthetic Modifying Agents

The composition preferably includes at least one aesthetic modifying agent. An aesthetic modifying agent is a material that imparts desirable tactile, olfactory, taste or visual properties to the surface to which the composition is applied. The aesthetic modifying agent may be hydrophobic or hydrophilic. The aesthetic modifying agent is preferably hydrophobic and is more preferably an oil, wax, solid or paste. Suitable aesthetic modifying agents include appropriate amounts of those materials suitable for solvents as defined above.

Other Adjuvants

Suitable adjuvants which may be incorporated into the base composition include pH adjusters, emollients, conditioning agents, moisturizers, chelating agents, gelling agents, colorants, fragrances, odor masking agents, UV stabilizer, preservatives, and any combination of any of the foregoing. Preferred pH adjusters include aminomethyl propanol, aminomethylpropane diol, triethanolamine, triethylamine, citric acid, sodium hydroxide, acetic acid, potassium hydroxide, lactic acid, and any combination of any of the foregoing.

Suitable conditioning agents include cyclomethicone; petrolatum; dimethicone; dimethiconol; silicone, such as cyclopentasiloxane and diisostearoyl trimethylolpropane siloxy silicate; sodium hyaluronate; isopropyl palmitate; soybean oil; linoleic acid; PPG-12/saturated methylene diphenyldiisocyanate copolymer; urea; amodimethicone; trideceth-12; cetrimonium chloride; diphenyl dimethicone; propylene glycol; glycerin; quaternary amines; and any combination of any of the foregoing.

The foregoing ingredients will be added to the compositions of the invention in effective amounts suitable for the intended use of the products and easily determined by those of skill in the art.

Methods of Preparing the Dispersions and Compositions of the Invention

The dispersions of the invention are preferably prepared using high pressure high shear processing. High pressure or high shear mixing maybe be performed in equipment which includes homogenizers such as a Microfluidizer, DeBee high pressure homogenizer, a french press and a Gaulin homogenizer or “Rotor Stator” devices such as a Symex mill, a Silverson mill and a Ross mill.

The dispersions of the invention may be formed by mixing together the components, including a wax (which is typically the active agent), an apolar solvent (such as silicone), water, PEG and/or butylene glycol, a preservative, and optionally one or more phospholipids or lecithins. The composition is heated (for example, to from about 50° C. to from about 80° C.) to melt the wax, then mixed with a propeller mixer and/or a homogenizer. The composition is then mixed and subjected to high pressure/high shear processing, to obtain a stable, homogeneous oil-in-water dispersion of the wax. The average pH of the composition is from about 5.0 to 5.5. The composition typically has a specific gravity of from about 0.8 to 1.00. The composition has an initial viscosity of from about 100 cps to greater than 50,000 cps. After 24 hours at 25° C., the viscosity of the composition is about 1,000 cps to greater than 80,000 cps.

In preferred embodiments, the wax and the hydrophobic solvent are first heated to the appropriate temperature to melt the wax. The wax and solvent are then added to any further hydrophobic components and the aqueous phase, and mixture subjected to high pressure/high shear processing to obtain a stable, homogeneous oil-in-water dispersion of the wax. The preferred pressure for preparation of this dispersion is between about 11,000 to about 27,000 psi, more preferably 11,000 to about 21,000 psi, most preferably between about 11,000 and 15,000 psi. The dispersion is stable for a commercially relevant period of time, e.g., between about 180 to 720 days when stored at approximate room temperature, in a commercial package.

The wax dispersions of the current invention may be used in the preparation of a finalized product for application by the end user. The wax dispersion can be added to a suitable base comprised of water and one or more Theological modifying agents and optionally a dispersion initiator. Although the composition may be used with surfactants, it is preferred that the composition contains no surface active agents to destabilize the dispersion. The wax dispersion may also be combined with other essentially surfactant-free dispersions comprised of one or more non-polar fluids to modify the textural and/or tactile properties of the finished goods product. Further, the wax dispersion can be combined with a physiologically active material to produce the performance benefit of the final finished goods product. The active ingredients can be either water soluble or not water soluble. Water soluble actives can be added to the water phase of the base while the water insoluble actives are first mixed into a hydrophobic-in-water dispersion then added to the base, the wax dispersions and the aesthetic modifying dispersions.

The following examples illustrate various aspects of the present invention. It should be understood that the invention is not limited to the specific examples or the details described therein.

### EXAMPLES

### Example 1

### Cocoa Butter Dispersion

Compositions 1A-1H were formed by heating the ingredients on a steam bath to approximately 80° C. The ingredients were mixed using a propeller mixer, followed by mixing in a homogenizer, and then subjected to a high pressure/high shear at from 11,000 to 25,000 psi. The components of compositions 1A-1H are shown in Table 1.

TABLE 1
Composition

Ingredient

| (wt %) | 1A | 1B | 1C | 1D | 1E | 1F | 1G | 1H |
|---|---|---|---|---|---|---|---|---|
| Cocoa Butter | 30.0 | 30.0 | 30.0 | 25.00 | 30.0 | 30.0 | 30.0 | 30.0 |
| USP¹ | | | | | | | | |
| (deodorized) | | | | | | | | |
| Silicone | 10.0 | 10.0 | 10.0 | 10.0 | 15.0 | 15.0 | 15.0 | 15.0 |
| (DC 345)² | | | | | | | | |
| PEG-4³ | 5.0 | — | 5.0 | 7.0 | 9.0 | 9.0 | — | 1.0 |
| PEG-8³ | — | 5.0 | — | — | — | — | — | — |
| Germazide ™ | 1.6 | 1.6 | 1.6 | 1.6 | 1.6 | 1.6 | 1.6 | 1.6 |
| MPB⁴ | | | | | | | | |
| Basis LP 20H⁵ | 2.8 | 2.8 | 2.8 | 2.7 | 3.0 | 1.4 | 1.5 | 1.6 |
| Alcolec ® BS⁶ | 0.6 | 0.6 | 0.75 | 0.7 | — | — | — | — |
| butylene | — | — | — | 1.0 | 2.5 | 2.5 | — | — |
| glycol⁷ | | | | | | | | |
| Deionized H₂O | 50.0 | 50.0 | 49.85 | 52.0 | 38.9 | 39.0 | 50.7 | 49.4 |
| Phos- | — | — | — | — | — | 1.5 | 1.2 | 1.4 |
| pholipon ® | | | | | | | | |
| 80H⁶ | | | | | | | | |

¹Jeen Chemical Company
²Dow Corning Corporation
³Dow Chemical
⁴Collaborative Laboratories, Inc.
⁵IKEDA
⁶American Lecithin
⁷KCL

### Example 2

### Shea Butter Extract

Compostions 2A-2I were formed by heating on a steam bath to approximately 80° C. a mixture of the ingredients listed in Table 2, and then mixing in a high shear mixer. The contents were then subjected to a high pressure/high shear processing at from 11,000 to 25,000 psi. The ingredients of compositions 2A-2I are shown below in Table 2.

TABLE 2
Composition

| Ingredient (wt %) | 2A | 2B | 2C | 2D | 2E | 2F | 2G | 2H | 2I |
|---|---|---|---|---|---|---|---|---|---|
| Shea Butter Extract¹ | 30.0 | 30.0 | 30.0 | 30.0 | 30.0 | 30.0 | 20.0 | 20.0 | 25.0 |
| PEG-8² | — | — | 10.0 | — | — | — | — | — | — |
| Silicone (DC 345)³ | 5.0 | 10.0 | — | 5.0 | 5.00 | — | 10.0 | 10.0 | 15.0 |
| Basis LP20H⁴ | 2.7 | 3.1 | 3.1 | 2.5 | 2.25 | 2.3 | 1.6 | 1.6 | 1.5 |
| Germazide ™ MPB⁵ | 1.5 | 1.5 | 1.5 | 1.5 | 1.50 | 1.5 | 1.6 | 1.6 | 1.6 |
| Deionized H₂O | 60.8 | 55.4 | 55.4 | 58.0 | 57.75 | 57.2 | 65.3 | 63.3 | 52.5 |
| Alcolec ® BS⁶ | — | — | — | 3.0 | 3.50 | 4.0 | — | 2.0 | 3.0 |
| butylene glycol⁷ | — | — | — | — | — | 5.0 | — | — | — |
| Phosopholipon 80H⁸ | — | — | — | — | — | — | 1.5 | 1.5 | 1.4 |

¹Fancor
²Dow Chemical
³Dow Corning Corporation
⁴IKEDA
⁵Collaborative Laboratories, Inc.
⁶American Lecithin
⁷KCL

### Example 3

### Cetearyl Alcohol Dispersion

Cetearyl Alcohol Dispersion was made by first heating a mixture of the ingredients shown in Table 3 in a steam bath to approximately 60° C. The ingredients were maintained at 60° C. while being mixed using a propeller mixer, followed by mixing in a homogenizer. Slowly the phospholipid was added, mixed until uniform and then subjected to high pressure/high shear processing at from 11,000 to 25,000 psi.

The processed material was transferred to a kettle with a side-swiping capability and mixed. 10% Butylene Glycol was added and the mixing with side-swipe action continued until the product was uniform. The product was then allowed to cool without further agitation.

TABLE 3

| Ingredient | Weight Percent |
|---|---|
| Deionized Water (0.2 μm filtered) | 59.76 |
| Germazide MPB¹ | 1.44 |
| Crodacol CS-50² | 8.10 |
| Hydrogenated Polyisobutene¹ | 18.90 |
| Basis LP20H³ | 1.80 |
| Butylene Glycol⁴ | 10.00 |

¹The Collaborative Group, Ltd.
²CRODA. Inc.
³IKEDA
⁴KCL

In order how to illustrate how the dispersions of the current invention can be used to prepare finished formulations, the experiments described in the following examples were conducted. All examples were prepared by first adding the dispersion described in Example 3 to either the Lotion Base or Moisturizing Base with propeller blade mixing. The remaining ingredients were all added sequentially and mixed with a paddle blade until completely uniform before adding the next ingredient.

### Example 4

### Moisturizing Cream for Very Dry Skin

TABLE 4

| Ingredient | Weight Percent |
|---|---|
| Moisturizing Base¹ | 55.3500 |
| Example 3 | 19.000 |
| Advanced Moisture Complex¹ | 4.7500 |
| Butylene Glycol² | 4.7500 |
| Seamollient¹ | 1.9000 |
| AM-400¹ | 9.5000 |
| AM-600¹ | 4.7500 |

¹Collaborative Laboratories, Inc.
²KCL

### Example 5

### Moisturizing Cream for Oily Skin

TABLE 5

| Ingredient | Weight Percent |
|---|---|
| Lotion Base¹ | 38.2700 |
| Example 3¹ | 8.4800 |
| Advanced Moisture Complex¹ | 4.2400 |
| Deionized Water | 17.6300 |
| Butylene Glycol² | 4.2400 |
| Sea Mollient¹ | 1.7000 |
| AM-100¹ | 8.4800 |
| AM-200¹ | 8.4800 |
| AM-300¹ | 8.4800 |

¹Collaborative Laboratories, Inc.
²KCL

### Example 6

### Moisturizing Cream for Sensitive Skin

TABLE 6

| Ingredients | Weight Percent |
|---|---|
| Lotion Base¹ | 35.85 |
| Example 3 | 4.7500 |
| Advanced Moisture Complex¹ | 4.7500 |
| Deionized Water | 23.9000 |
| AM-300¹ | 9.5000 |
| AM-400¹ | 9.5000 |
| AM-500¹ | 9.5000 |
| Sea Parsley¹ | 0.2500 |
| Sansurf ™ Bisabolol¹ | 2.0000 |

¹Collaborative Laboratories, Inc.

### Example 7

### SPF 15 Moisturizing Cream

TABLE 7

| Ingredients | Weight Percent |
|---|---|
| Moisturizing Base¹ | 44.7100 |
| Example 3 | 8.2600 |
| Advanced Moisture Complex¹ | 4.1300 |
| Dionized Water | 0.6000 |
| Butylene Glycol² | 4.1300 |
| Seamollient¹ | 1.6500 |
| AM 600¹ | 8.2600 |
| AM 700¹ | 8.2600 |
| Solarease II¹ | 20.0000 |

¹Collaborative Laboratories, Inc.
²KCL

### Example 8

### Anti-aging Cream

TABLE 8

| Ingredients | Weight Percent |
|---|---|
| Moisturizing Base¹ | 44.7000 |
| Example 3 | 8.2600 |
| Advanced Moisture Complex¹ | 4.1300 |
| Butylene Glycol² | 4.1300 |
| Seamollient¹ | 1.6500 |
| AM-600¹ | 8.0200 |
| AM-700¹ | 8.0200 |
| Coll-Age¹ | 0.0100 |
| Scavenol¹ | 0.0400 |
| AHA Liposomes¹ | 0.2400 |
| Micromerol¹ | 0.4000 |
| Phytosol Heather¹ | 0.4000 |
| Solarease II¹ | 20.0000 |

¹Collaborative Laboratories, Inc.
²KCL

What is claimed is:

1. A method for preparing a surfactant-free oil-in-water wax dispersion, comprising the steps of:
(a) mixing a wax with one or more hydrophobic components to form a hydrophobic phase; and
(b) mixing the hydrophobic phase with an aqueous phase under conditions of high shear/high pressure to form an oil in water wax dispersion.

2. The method of claim 1 wherein the wax is present in an amount of from 1.0 to 50.0 wt % of the total dispersion and preferably from 5.0 to 40.0 wt %.

3. The method of claim 1 wherein the hydrophobic phase is present in an amount of from 1.0 to 60.0 wt % of the total dispersion and preferably from 5.0 to 40.0 wt %.

4. The method of claim 1 wherein the aqueous phase is present in an amount of from 40.0 to 99.0 wt % of the total dispersion and preferably from 60.0 to 95.0 wt %.

5. The method of claim 1 wherein the dispersion has a specific gravity of 0.8 to 1.0.

6. The method of claim 1, wherein the wax is selected from the group consisting of shea butter, cocoa butter, carnula wax, petrolatum, castor wax, chamomile wax, bayberry wax, candelilla wax, carnauba wax, clary wax, beeswax, orange peel wax, Japan wax, mink wax, rice bean wax, oricury wax, palm kernel wax, raspberry leaf wax, rosemary flower wax, sweet violet leaf wax, tuberose flower wax, ylang ylang flower wax, everlasting wax, myrtle leaf wax, spermaceti wax, jojoba wax, jotoba butter waxes, and combinations thereof.

7. The method of claim 1, wherein the hydrophobic components are selected from the group consisting of hydrogenated castor oil, hydrogenated cottonseed oil, hydrogenated jojoba oil, hydrogenated palm kernel oil, hydrogenated rapeseed oil and hydrogenated rice bran wax.

8. The method of claim 1, wherein the wax is selected from the group consisting of ceresine wax, ozokerite, paraffin wax and microcrystalline wax.

9. The method of claim 1, wherein the wax is selected from the group consisting of synthetic beeswax, synthetic candelilla wax, synthetic carnauba wax, or synthetic spermaceti.

10. The method of claim 1, wherein the wax is selected from the group consisting of cetyl demethicone, dialkyldimethyl polysiloxane, dimethiconol hydroxystearate, dimethiconol stearate, stearoxymethicone/dimethicone copolymer and stearoxytrimethylsilane.

11. The method of claim 1, wherein the hydrophobic phase further comprises a plasticizing agent.

12. The method of claim 11 wherein the plasticizing agent is present in an amount of from 0.5 to 30.0 wt % of the total dispersion and preferably from 1.0 to 20.0 wt %.

13. The method of claim 11, wherein the plasticizing agent is selected from the group consisting of cyclomethicone, hydrogenated polyisobutene, polyethylene glycol fluid and butylene glycol.

14. The method of claim 1, wherein the hydrophobic phase further comprises a solvent, and optionally a co-solvent.

15. The method of claim 14, wherein the solvent is present in an amount of from 5.0 to 50.0 wt % of the total dispersion and preferably from 10.0 to 40.0 wt %.

16. The method of claim 14, wherein the co-solvent is present in an amount of from 1.0 to 25.0 wt % of the total dispersion and preferably from 2.0 to 20.0 wt %.

17. The method of claim 14, wherein the solvent or co-solvent is selected from the group consisting of mineral oil, petrolatum, polybutylene and hydrogenated polyisobutene.

18. The method of claim 14, wherein the solvent or co-solvent is propylene glycol monoisostearate.

19. The method of claim 14, wherein the solvent or co-solvent is selected from the group consisting of soybean oil, babassu oil, castor oil, cottonseed oil, Chinese tallow oil, crambe oil, perilla oil, Danish rapeseed oil, rice bran oil, palm oil, palm kernel oil, olive oil, linseed oil, coconut oil, sunflower oil, safflower oil, peanut oil, and corn oil.

20. The method of claim 14, wherein the solvent or co-solvent has the formula: 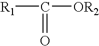
wherein R₁ and R₂ are saturated, unsaturated, branched or cyclic alkyl radicals of 2 to 24 carbon atoms and n is an integer from 0 to about 20.

21. The method of claim 14, wherein the solvent or co-solvent has the formula: 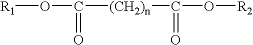
wherein R₁ and R₂ are saturated, unsaturated, branched or cyclic alkyl radicals of 2 to 24 carbon atoms and n is an integer from 0 to about 20.

22. The method of claim 14, wherein the solvent or co-solvent has the formula: 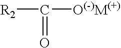
wherein R₁ is a saturated, unsaturated, branched or cyclic alkyl radical from C₂ to C₂₄; M⁽⁺⁾ is defined by NR₂R₃R₄R₅ where R₂, R₃ and R₄ are hydrogen or a saturated, unsaturated or branched alkyl or hydroxalkyl radical from C₁ to C₁₀; R₅ is a saturated, unsaturated, branched or cyclic alkyl or substituted alkyl radical from C₂ to C₂₄.

23. The method of claim 22, wherein the solvent is lauramine oleate.

24. The method of claim 14, wherein the solvent or co-solvent has the formula: 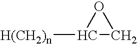
wherein n is an integer from 0 to about 3.

25. The method of claim 24, wherein the solvent or co-solvent is selected from the group consisting of polyethylene oxide, polypropylene oxide and polybutylene oxide.

26. The method of claim 14, wherein the solvent or co-solvent is selected from the group consisting of cyclomethicone, dimethicone polysiloxane, dimethiconol, polyalkylsiloxanes, polyarylsiloxanes, polyalkylarylsilanes, polyalkylarylsiloxanes, polysiloxanes and polysiloxane copolymers.

27. The method of claim 26, wherein the solvent is dimethicone or phenyltrimethicone.

28. The method of claim 1, wherein the aqueous phase further comprises a dispersion initiator.

29. The method of claim 28 wherein the dispersion initiator is present in an amount of from 0.01 to 10.0 wt % of the total dispersion and preferably from 0.1 to 5.0 wt %.

30. The method of claim 28, wherein the dispersion initiator is selected from the group consisting of phospholipid, lecithin, and combinations thereof.

31. A surfactant-free oil-in-water wax dispersion prepared by the method of claim 1.

32. The dispersion of claim 31 wherein the wax is present in an amount of from 1.0 to 50.0 wt % of the total dispersion and preferably from 5.0 to 40.0 wt %.

33. The dispersion of claim 31 wherein the hydrophobic phase is present in an amount of from 1.0 to 60.0 wt % of the total dispersion and preferably from 5.0 to 40.0 wt %.

34. The dispersion of claim 31 wherein the aqueous phase is present in an amount of from 40.0 to 99.0 wt % of the total dispersion and preferably from 60.0 to 95.0 wt %.

35. The dispersion of claim 31 wherein the dispersion has a specific gravity of 0.8 to 1.0.

36. The dispersion of claim 31, wherein the wax is selected from the group consisting of shea butter, cocoa butter, carnula wax, petrolatum, castor wax, chamomile wax, bayberry wax, candelilla wax, carnauba wax, clary wax, beeswax, orange peel wax, Japan wax, mink wax, rice bean wax, oricury wax, palm kernel wax, raspberry leaf wax, rosemary flower wax, sweet violet leaf wax, tuberose flower wax, ylang ylang flower wax, everlasting wax, myrtle leaf wax, spermaceti wax, jojoba wax, jotoba butter waxes, and combinations thereof.

37. The dispersion of claim 31, wherein the hydrophobic components are selected from the group consisting of hydrogenated castor oil, hydrogenated cottonseed oil, hydrogenated jojoba oil, hydrogenated palm kernel oil, hydrogenated rapeseed oil and hydrogenated rice bran wax.

38. The dispersion of claim 31, wherein the wax is selected from the group consisting of ceresine wax, ozokerite, paraffin wax and microcrystalline wax.

39. The dispersion of claim 31, wherein the wax is selected from the group consisting of synthetic beeswax, synthetic candelilla wax, synthetic carnauba wax, or synthetic spermaceti.

40. The dispersion of claim 31, wherein the wax is selected from the group consisting of cetyl demithicone, dialkyldimethyl polysiloxane, dimethiconol hydroxystearate, dimethiconol stearate, stearoxymethicone/dimethicone copolymer and stearoxytrimethylsilane.

41. The dispersion of claim 31, wherein the hydrophobic phase further comprises a plasticizing agent.

42. The dispersion of claim 31, wherein the plasticizing agent is present in an amount of from 0.5 to 30.0 wt % of the total dispersion and preferably from 1.0 to 20.0 wt %.

43. The dispersion of claim 31, wherein the plasticizing agent is selected from the group consisting of cyclomethicone, hydrogenated polyisobutene, polyethylene glycol fluid and butylene glycol.

44. The dispersion of claim 31, wherein the hydrophobic phase further comprises a solvent, and optionally a co-solvent.

45. The dispersion of claim 44, wherein the solvent is present in an amount of from 5.0 to 50.0 wt % of the total dispersion and preferably from 10.0 to 40.0 wt %.

46. The dispersion of claim 44, wherein the co-solvent is present in an amount of from 1.0 to 25.0 wt % of the total dispersion and preferably from 2.0 to 20.0 wt %.

47. The dispersion of claim 44, wherein the solvent or co-solvent is selected from the group consisting of mineral oil, petrolatum, polybutylene and hydrogenated polyisobutene.

48. The dispersion of claim 44, wherein the solvent or co-solvent is propylene glycol monoisostearate.

49. The dispersion of claim 44, wherein the solvent or co-solvent is selected from the group consisting of soybean oil, babassu oil, castor oil, cottonseed oil, Chinese tallow oil, crambe oil, perilla oil, Danish rapeseed oil, rice bran oil, palm oil, palm kernel oil, olive oil, linseed oil, coconut oil, sunflower oil, safflower oil, peanut oil, and corn oil.

50. The dispersion of claim 44, wherein the solvent or co-solvent has the formula: 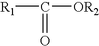
wherein R₁ and R₂ are saturated, unsaturated, branched or cyclic alkyl radicals of 2 to 24 carbon atoms and n is an integer from 0 to about 20.

51. The dispersion of claim 44, wherein the solvent or co-solvent has the formula: 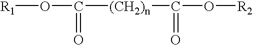
wherein R₁ and R₂ are saturated, unsaturated, branched or cyclic alkyl radicals of 2 to 24 carbon atoms and n is an integer from 0 to about 20.

52. The dispersion of claim 44, wherein the solvent or co-solvent has the formula: 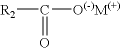
wherein R₁ is a saturated, unsaturated, branched or cyclic alkyl radical from C₂ to C₂₄; M⁽⁺⁾ is defined by NR₂R₃R₄R₅ where R₂, R₃ and R₄ are hydrogen or a saturated, unsaturated or branched alkyl or hydroxalkyl radical from C₁ to C₁₀; R₅ is a saturated, unsaturated, branched or cyclic alkyl or substituted ally1 radical from C₂ to C₂₄.

53. The dispersion of claim 52, wherein the solvent is lauramine oleate.

54. The dispersion of claim 44, wherein the solvent or co-solvent has the formula: 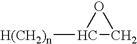
wherein n is an integer from 0 to about 3.

55. The dispersion of claim 54, wherein the solvent or co-solvent is selected from the group consisting of polyethylene oxide, polypropylene oxide and polybutylene oxide.

56. The dispersion of claim 44, wherein the solvent or co-solvent is selected from the group consisting of cyclomethicone, dimethicone polysiloxane, dimethiconol, polyalkylsiloxanes, polyarylsiloxanes, polyalkylarylsilanes, polyalkylarylsiloxanes, polysiloxanes and polysiloxane copolymers.

57. The dispersion of claim 56, wherein the solvent is dimethicone or phenyltrimethicone.

58. The dispersion of claim 31, wherein the aqueous phase further comprises a dispersion initiator.

59. The dispersion of claim 58, wherein the dispersion initiator is present in an amount of from 0.01 to 10.0 wt % of the total dispersion and preferably from 0.1 to 5.0 wt %.

60. The dispersion of claim 58, wherein the dispersion initiator is selected from the group consisting of phospholipid, lecithin, and combinations thereof.

61. A composition comprising:
a) an oil in water dispersion prepared by the method of claim 1; and
b) a base composition comprising
(i) a hydrophilic Theological modifying agent, and
(ii) an aqueous phase.

62. The composition of claim 61 wherein the base composition comprises from about 0.01 to about 10% by weight of hydrophilic Theological modifying agent.

63. The composition of claim 61 wherein the base composition comprises from about 20 to about 99.99% by weight of water.

64. The composition of claim 61 wherein the dispersion comprises from about 0.1 to 80% by weight of the total composition.

65. The composition of claim 61 wherein the base composition comprises from about 10 to about 99% by weight of the total composition.

66. The composition of claim 61, wherein the hydrophilic Theological modifying agent comprises a hydrophilic gelling agent.

67. The composition of claim 66, wherein the hydrophilic gelling agent comprises one or more members selected from the group consisting of carboxyvinyl polymers, acrylic copolymers, polyacrylamides, polysaccharides, natural gums and clays.

68. The composition of claim 61, wherein the Theological modifying agent comprises a phosphorylated starch derivative.

69. The composition of claim 68, wherein the phosphorylated starch derivative is hydroxypropyl distarch phosphate.

70. The composition of claim 61, wherein the Theological modifying agent is selected from the one or more members of the group consisting of sodium hyaluronate, acrylates/C₁₀-C₃₀ alkyl acrylate crosspolymer, xanthum gum, cholesterol, hydroxypropyl distarch phosphate, carbomer, guar hydroxy propyltrimonium chloride, hydroxypropyl guar and sodium hydroxypropyl starch phosphate.

71. The composition of claim 61 further comprising anti-acne agents, antimicrobial agents, anti-inflammatory agents, analgesics, antierythemal agents, antipruritic agents, antiedemal agents, antipsoriatic agents, antifungal agents, skin protectants, sunscreen agents, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, amino acids, peptides, minerals, ceramides, biohyaluronic acids, enzymes and any combination of any of the foregoing

72. The composition of claim 61, further comprising aesthetic modifying agents.

73. The composition of claim 61 for topical, anal, vaginal, ophthalmic, nasal or otic application.

74. The composition of claim 61 in the form of a lotion, cream, paste, serum or spray.
